(19) United States
(12) Patent Application Publication
(10) Pub. No.: US 2003/0087334 A1
(43) Pub. Date: May 8, 2003

(54) Method of flexibly generating diverse reaction chemistries

(75) Inventors: Barry A. Bunin, Santa Clara, CA (US); Timothy S. Powers, Oakland, CA (US); Guillermo Antonio Morales, Patterson, CA (US); Stephan C. Schurer, Mountain View, CA (US); Steve M. Muskal, Carlsbad, CA (US); Oliver L. Saunders, Burlingame, CA (US)

Correspondence Address: BEYER WEAVER & THOMAS LLP, P.O. BOX 778, BERKELEY, CA, 94704-0778, US

(73) Assignee: Libraria, Inc.

(21) Appl. No.: 10/300,730

(22) Filed: Nov. 19, 2002

Related U.S. Application Data
(63) Continuation-in-part of application No. 09/825,135, filed on Apr. 2, 2001.
(60) Provisional application No. 60/332,230, filed on Nov. 20, 2001.

(51) Int. Cl. C12P 1/00

(52) U.S. Cl. 435/41

(57) ABSTRACT

Computational tools automatically suggest/generate diverse reaction sets for particular precursors, classes of precursors, or different reaction chemistries. This is accomplished by automatically generating a group of reaction chemistries for a particular precursor or class of precursors. Some of the reactions and/or products may be produced without reliance on reactions and products reported in available references.

### CROSS REFERENCE TO RELATED APPLICATIONS

The application claims priority under 35 U.S.C. 119(e) from U.S. Provisional Patent Application No. 60/332,230 (filed on Nov. 20, 2001 by Bunin et al., and titled “Method Of Flexibly Generating Diverse Reaction Chemistries”) and this application is a continuation-in-part of U.S. patent application Ser. No. 09/825,135 (filed on Apr. 2, 2001 by Barry A. Bunin, and titled “Chemistry Resource Database”). Both of these patent applications are incorporated herein by reference in their entireties and for all purposes. This application is related to U.S. patent application Ser. No. 09/996,635, filed on Nov. 28, 2001 by Bunin et al. and titled “Chemistry Resource Database.” That application is incorporated herein by reference in its entirety and for all purposes.

### FIELD OF THE INVENTION

The invention relates to database technology. More specifically, the invention relates to software tools for generating diverse reaction sets from particular precursors, classes of precursors, or reaction chemistries.

### BACKGROUND OF THE INVENTION

The modern organic chemist has numerous software tools at her disposal. These include tools for predicting activity from chemical structure (termed “structure activity relationship” tools or SAR tools), tools for ordering commercially available reagents, and databases for storing vast quantities of chemical information including links to literature. Many of these tools have appeared recently in order to take advantage of new electronic infrastructure and electronic commerce. Others have appeared because the computational power now exists to solve previously intractable problems (or reasonably approximate a solution to these problems).

Some of the most widely used on-line databases provide electronically indexed data that previously appeared in textual research tools on library shelves (e.g., Beilstein, Chemical Abstracts, and the like). While such databases include various modern electronic features, they are at their heart collections of traditional chemical information reformatted for electronic databases. These existing databases are essentially lists indexing the literature with information to help the chemist decide if she wishes to obtain a particular article. As such they are not optimized to facilitate the research of a modern chemist.

One set of problems that cannot be easily addressed using current chemical software pertains to constraints on the vast range of reaction conditions available to a chemist. Another important issue is access to detailed information on reactivity and chemical pathways in a database format, especially for high-throughput chemistry. Often the inherent features of a laboratory facility or piece of chemical instrumentation will constrain the range of reaction conditions available to the chemist. A good example is found in combinatorial chemistry or parallel synthesis laboratory equipment. Commonly, such apparatus have performance constraints, and thus are unable to provide a wide range of reaction conditions for chemical synthesis. This is because such apparatus must be designed to perform many chemical syntheses simultaneously and on varying reaction scale. Hence the apparatus are quite intricate. This can make it difficult for such apparatus to handle variable heating and cooling, inert atmospheres, highly reactive reagent delivery, etc. Furthermore, many interesting molecules are unstable to conditions such as heat and/or light. If a chemist uses a software tool to suggest compounds with desired properties and potentially useful activities, she would like to know how stable such compounds are (and if they can in fact be synthesized) with the support (infrastructure and other) available to her. Currently, no tool provides such information. Part of the problem is that current databases do not effectively organize and provide chemical information based on chemical reaction conditions or reaction chemistries.

Another potential problem arises when a chemist undertakes a new line of research and needs to use unfamiliar synthesis chemistries. In such cases, she would probably like to start with compounds that are relatively easy to synthesize. Often within a class of precursors that undergo a particular reaction, some members will undergo the reaction much more reliably and effectively than others. The chemist new to this field will wish to know which such precursors are most reliable. Similarly, an experienced chemist often comes upon reactions that intuitively should work, but in practice do not. Reaction chemistries coupled with reliability ratings can provide a powerful tool to the synthetic chemist, saving time and money which would otherwise be wasted on fruitless experiments. Unfortunately current software tools fail to conveniently address and provide reliability data to the chemist.

Yet another problem confronts chemists attempting to generate diverse libraries of compounds for drug discovery or other product discovery research. The literature and databases are limited in their ability to suggest the full potential of a discovery path. Often a chemist will understand that some portion of a compound (a compound fragment) is at least partially responsible for a desired activity or other property. Such knowledge may derive from pharmacophore research, for example. In the discovery process, the chemist will wish to generate a library of compounds possessing the fragment of interest or some variant thereof. To do so, she may use combinatorial chemistry (parallel synthesis) to generate a large library of compounds. In combinatorial chemistry multiple precursors, each having the desired fragment, are further elaborated through one or more syntheses. The resulting library of compounds is diverse but limited by the reaction chemistries either known to the chemist or found by searching through conventional databases. Greater diversity could be achieved if additional variations on reaction chemistry, not necessarily in the literature, were suggested to the chemist.

The problems resulting from the above software limitations are compounded by the ever increasing pace of chemical research. New synthetic procedures are developed, tested, and retested daily. New insights into organic chemistry, structural biology, and drug discovery occur frequently. It is a mighty challenge for electronic repositories of chemical information to keep pace with these developments.

What is needed are improved software tools for the research chemist that facilitate chemical research in general and drug discovery in particular.

### SUMMARY OF THE INVENTION

The present invention addresses these needs by providing improved software tools that employ databases and associated systems for storing, manipulating, and investigating chemical information organized by reaction chemistries and/or transformations. At least some reaction chemistries are organized as belonging to particular reaction protocols within the database. Each reaction represents a discrete step in a multi-step protocol for making a final product from a starting reactant. The present invention uses chemical and reaction databases having information stored such that individual molecules, reactions, and protocols are tagged according to many criteria. Using these tags, software of the invention can not only logically retrieve information, but also use logic to extrapolate from known chemistries in order to provide the user with valuable synthetic information.

The invention provides software tools that help automate suggestions of and generation of diverse reaction sets for particular precursors, classes of precursors, or different reaction chemistries. This is accomplished by automatically generating a group of reaction chemistries for a particular precursor or class of precursors of relevance for diverse problems of commercial and scientific interest. Some of the reactions and/or products may be produced without reliance on reactions and products reported in available references, for example the chemical information databases mentioned above.

Thus, one aspect of the invention is a method of identifying, from a database, a collection of chemical compounds that can be synthesized from a common reactant or class of reactants. Such methods may be characterized by the following sequence: (a) automatically generating a list of reactions that the class to which the reactant or reactants belong undergoes; and (b) for each reaction in the list, identifying a product or class of products, which products comprise the collection of chemical compounds. Preferably, at least some of the products are identified in or inferred from the database, which contains reactions reported in references, and at least some of the products are identified without reliance on reactions reported in references.

Additionally, such methods may further include: (c) treating at least one of the products as a new reactant; (d) automatically generating a new list of reactions that the class to which the new reactant belongs; and (e) for each reaction in the new list, identifying a new product or class of products produced by the reaction. Preferably the new products are added to the collection of compounds.

Alternatively, such methods may include filtering the products identified at (b) based on one or more of the following criteria: predicted activity and specified reaction conditions.

Another aspect of the invention is a method of identifying a collection of chemical compounds that can be synthesized from a common reactant or class of reactants. Such methods can be characterized by the following sequence: (a) automatically generating a list of reactions that the class to which the reactant or reactants belong undergoes; (b) for each reaction in the list, identifying a product or class of products; and (c) filtering the products identified in (b) to yield a subset of the compounds identified (b), which subset comprises the collection of chemical compounds. Preferably, the filtering is based on one or more of the following criteria: predicted activity, number of reaction steps required to produce the product, specified reaction conditions, and relevance of the final products enumerated. Also preferably, at least some of the products are identified in a database containing reactions reported in references and at least some of the products are identified without reliance on reactions reported in references and generating the list of reactions is accomplished, at least in part, without reliance on reactions reported in references.

Additionally, such methods may further include: (d) treating at least one of the compounds in the subset as a new reactant; (e) automatically generating a new list of reactions that the class to which the new reactant belongs; and (f) for each reaction in the new list, identifying a new product or class of products produced by the reaction. Preferably, the new products are added to the collection of compounds.

Another aspect of the invention pertains to methods of identifying biologically active compounds based on the chemical structure of a known biologically active compound. The method may be characterized by the following sequence: (a) retrosynthetically decomposing the known biologically active compound into two or more building blocks; (b) identifying multiple chemical reactions that a first one of the building blocks can undergo; (c) identifying, with the aid of a chemical database, products of said multiple chemical reactions; (d) identifying a potentially biologically active compound by linking at least one of said products with one or more of the other building blocks, whether transformed or not; and (e) conducting a computational screen to predict whether the potentially biologically active compound is likely to be biologically active. As examples, the screen may be a pharmacophore screen or a docking algorithm.

Yet another aspect of the invention pertains to computer program products including machine-readable media on which are provided program instructions for implementing the methods described above, in whole or in part. Frequently, the program instructions are provided as code for performing certain method operations. Any of the methods of this invention may be represented, in whole or in part, as program instructions that can be provided on such machine-readable media. In addition, the invention pertains to various combinations and arrangements of data generated and/or used as described herein.

These and other features and advantages of the present invention will be described in more detail below with reference to the associated figures.

### BRIEF DESCRIPTION OF THE DRAWINGS

FIG. 1A is a block diagram depicting how logic of the invention can generate a novel reaction sequence.

FIG. 1B is a synthetic scheme depicting how logic of the invention can provide the user with reaction sequences for maximizing diversity.

FIG. 2 is a process flow diagram depicting a general methodology for using a database of chemical/biological information to facilitate design of chemical structures having a desired biological activity in accordance with an embodiment of this invention.

FIG. 3A is a structural depiction of the Gleevec™ molecule.

FIGS. 3B-3C depict, structurally, a suitable retrosynthetic analysis performed on the Gleevec™ molecule.

FIGS. 3D-3E depict and exemplary Gleevec™ building block transformation in accordance with an example of this invention.

FIG. 3F depicts, for the sake of illustration, various synthetic pathways that are available to a generic aldehyde constituent.

FIG. 3G depicts an example Gleevec™ analog generated from transformed building blocks in accordance with an embodiment of this invention.

FIG. 4A shows a sample screen shot from a database application used with an example of this example.

FIG. 4B depicts a process employed to generate a pharmacophore for use in an example of this invention.

FIG. 5A presents an example subset of possible chemistry patterns that illustrate the flexible synthetic analysis that can be performed by the present invention.

FIG. 5B shows how the invention may used to replace the amide bond in Statine inhibitors.

FIG. 6 illustrates, in simple block format, a typical computer system that, when appropriately configured or designed, can serve as a computational apparatus of this invention

### DETAILED DESCRIPTION OF THE PREFERRED EMBODIMENTS

In the following detailed description of the present invention, numerous specific embodiments are set forth in order to provide a thorough understanding of the invention. However, as will be apparent to those skilled in the art, the present invention may be practiced without these specific details or by using alternate elements or processes. In some descriptions herein, well-known processes, procedures, and components have not been described in detail so as not to unnecessarily obscure aspects of the present invention.

Definitions

To clearly describe certain embodiments of the invention, some terms used herein are defined as follows. These definitions are provided to assist the reader in understanding the concepts exemplified in the specification and the definitions do not necessarily limit the scope of this invention.

Reaction—A “reaction” is a fundamental chemical transformation of one or more reactants to one or more products. As used herein “reaction” and “reaction step” are synonymous. Examples of reactions include condensation reactions (e.g., esterification, amidation, imine formation), carbon-carbon couplings (e.g., Suzuki, Wittig, Heck), and reduction reactions (e.g., nitro reductions, hydrogenations, reductive amination). Multiple reactions may be concatenated to produce a “protocol” or synthetic pathway to a final product. To succeed, a reaction may require certain reaction conditions. Such conditions may include a reaction temperature, a reaction time, etc. Specialized laboratory instrumentation may be required to provide the needed reaction conditions. Commonly, a reaction will employ one or more reagents and/or solvents.

Protocol—A protocol is a group of chemical reactions, typically performed sequentially, to carry out an encompassing transformation from a starting reactant or reactants to a final product or products. Such sequential reactions may be carried out in parallel and converge at some point to a product or products. The terms “reaction scheme” and “synthetic pathway” are often used in the art to mean “protocol,” as that term is used herein. A protocol may include not only its constituent reactions, but also any associated reaction conditions used to carry out each of the reactions or reaction steps. An example of a multi-step protocol is a synthesis of a particular tripeptide using sequential two reactions. The first reaction couples a first amino acid to a second amino acid to form a dipeptide. The second reaction couples the dipeptide to a third amino acid to form the tripeptide.

Reference—A reference is document or other medium containing pertinent information. In the context of this invention, the pertinent information is usually chemical information. This concept includes traditional published literature articles, published and unpublished patent documents (patent applications and issued patents), unpublished experimental results, books, monographs, abstracts, and the like.

Reactant—This term encompasses the compounds used in any particular reaction that are transformed or converted by the reaction to a product. Specifically for this invention, reactants are those molecules that are modified in some way to become part of or are incorporated into the product molecule or molecules of a reaction, and thus are not “spectators” in the reaction.

Reagent—Reagents are those compounds used in a reaction that ultimately do not end up as part of the product molecules. Such molecules include solvents, catalysts, and other reaction mediators. Reagents are, overall, spectators in the reaction. Although they may be intimately involved with the reactants during the reaction, generally neither they nor subsets of their molecular structure become incorporated into the product's molecular structure. Generally solution phase reactions refer to homogeneous reactions; however, some solution phase reactions referred to are heterogeneous reactions. A reagent used in a solution phase reaction may mediate the reaction while immobilized on a solid phase support or may itself exist in the solid phase. For example an isocyanate scavenger bound to an inert polymer resin may be used to trap excess amine in a solution phase reaction or a catalyst solid may itself remain as a solid in a solution phase reaction. For solid phase reactions, generally one reactant is immobilized on a solid support medium, and other reactants and “reagents” used in the reaction are in the liquid phase. For example, a “scaffold” or “template” molecule is immobilized on a solid support. A reaction with this molecule is then performed in a particular solvent (reagent) with one or more reactants, parts of which become integrated into the scaffold or template molecule to become part of a product molecule, itself still bound to the solid support. The product molecule is then freed from the solid support using a “cleaving reagent,” an intramolecular rearrangement, or other technique such as irradiative cleavage of a linker-product bond.

Solvent—A solvent is generally the liquid medium in which a reaction takes place. For solid-phase reactions, a solvent is the liquid medium in which solid phase supported reactants are suspended and reagents are dissolved. For solution phase reactions, a solvent is the liquid medium in which reactants are dissolved, but solid phase reagents are suspended. There are cases in which a compound serves multiple roles, for example as both solvent and reactant or both solvent and reagent.

Reaction condition—Reaction condition refers to parameters under which a reaction takes place, for example, time, temperature, pressure, radiation, solvents or reagents used.

Laboratory instrumentation or equipment—These terms refer to the hardware used to carry out reactions; i.e. for traditional synthesis any glassware, heating devices, pressure vessels etc. and for combinatorial or parallel synthesis any hardware used to perform multiple reactions in parallel. Sometimes this term is used to define the minimal amount of hardware necessary to carry out a reaction; i.e., hardware that does not include peripheral devices or equipment not crucial to carry out a reaction. In this context, the term might not encompass a particular robotic device, for example.

Procedure—Generally, a procedure is a “recipe” for a particular chemical transformation. More specifically, a procedure refers to the detailed methods used by the chemist to carry out a reaction. Typically a procedure refers to a detailed textual account of the sequence of events, reagents, reactants, laboratory instrumentation or equipment, and reaction conditions used by the chemist to carry out a particular reaction. A procedure then allows a chemist in a laboratory to reproduce the chemical reaction to which the procedure refers without access to other source of information regarding the reaction being carried out.

Product—Products are molecules that result from a reaction of reactants. Thus for example, a chemist using a set of reactants and using associated procedures converts the reactants into a product or set of products.

Ontology—Ontology in this application refers to the logical linkage of categories used to classify a type of chemical information such as a reference, a protocol, or a reaction. These categories are often arranged in levels of a hierarchy. For example, a reference may be categorized at a high level as pertaining to either solid-phase chemistry or solution-phase chemistry. Each of these categories may be further categorized as to the reaction type, for example condensation reaction, carbon-carbon bond forming reaction, substitution reaction, and the like. Still further, each of these categories is further categorized more definitively, for example a condensation reaction category may contain amide-forming condensations, ester-forming condensations, imine-forming condensations, and the like. Each of references, protocols, and reactions possess hierarchical components of a given ontology.

Enumeration—Often a compound or reaction is represented in a generic format. That is, for a particular compound reaction genus there may be multiple species. A generic compound (reactant or product) is represented as a core structure having one or more substituents represented generically, as for example an “R-group.” For a given R-group, there are a number of particular chemical moieties that define distinct species of the generic compound. A generic compound is “enumerated” by displaying or otherwise identifying the species comprising the genus. Each species represents a specific compound containing the core structure and a specific chemical moiety at the location of each R-group. Stated another way, enumeration refers to electronically reconstructing representations of the actual structures (reactants and products) for each species reaction. For example, a reaction employing 5 amines and 5 carboxylic acids under dehydrating conditions would generate 25 amides through enumeration. Obviously, the concept of enumeration can extend to groups of generic chemical compounds, as one might encounter in a generic representation of a reaction. In one example, a reference identifies 100 reactions that were carried out, each reaction being a species of a generic reaction. In one format, the reference may depict only the generic reaction. When enumerated, all 100 specific reactions are depicted. In some embodiments of this invention, it will be convenient to separately store in electronic format the actual R-group moieties used.

Chemical Information—this includes all information in a reference, database, or other medium that pertains to a chemical compound, a chemical reaction, collections of compounds or reactions, and the like. The chemical information may be provided in various textual, numerical, and/or structural formats. Often the information will include pertinent annotations such as reaction conditions, laboratory instrumentation, solvents, reagents, details about a reference, etc.

Filter—Generally, a filter refers to a constraint applied to a search in order to narrow or more fully define the search. In the context of this invention, a filter can be any number of search constraints that are added to a search query or applied to a set of results from such a query to further narrow or define the result in terms of the particular filter or filters applied. Filters used in embodiments of the invention can be applied at the reference, protocol, and/or reaction level as well as any fields that are contained in records of databases of the invention. Data can be searched and filtered in many combinations of ways. Examples of filters include, but are not limited to, the following: reaction condition, reaction type, library size, number of steps in a protocol, yield of reaction, molecular weight, reactant type, logP, ADMET/PK, Lipinski's rule of five, QSAR, pharmacophore, docking, binding, structure, substructure, reliability ratings, biological activity, reactivity, starting material, product, author, journal, keywords, vendor, leading references, and the like.

Database—A set of related files or records that is created and managed by a database management system. The records may include text, images, sound, video, etc. A record is a group of related fields that store data about a subject or activity.

Databases Organized by Chemical Synthesis Methods

To give users great flexibility in querying by reaction chemistry and process conditions, the invention provides databases of chemical information organized by chemical synthesis methods. Generally, this means organization by reaction type. Preferably, though not necessarily, these databases are relational databases. In the databases of this invention, chemical reactions are classified according to type, reaction information, specific aspects of procedures and methods used in the reaction, product yield, reliability rating, and chemical reagents are classified according to functional group and compatible synthetic methods. In some examples, specific chemical reaction/process information is used as primary or foreign keys in relational database tables. In fact, the primary key of some database tables may be a combination of reaction type (e.g., reductive amination) and either a reactant or a product. Still further, the database keys may comprise particular reaction conditions (e.g. temperature ranges, solvent classes, pressure ranges, etc.) in association with reaction type. Beyond this, chemical information can be organized by biological activity such as Ki or IC50 values for interactions with particular biological targets. At a minimum, reaction types, biological activity, and/or reaction conditions may be provided as attributes or columns of individual database records. Those of skill in the art will understand that numerous database schemas may be used to implement the functionality described below.

Conventional chemical database search engines provide examples of reaction types based on user queries. For example, if a user queries an esterification reaction generically (typically by structure or substructure), conventional systems normally provide a list of esterification reactions from the literature in no particular order or rank. More constrained queries can be input to provide more relevant examples, and hopefully reasonably short lists. Unfortunately, by putting more stringent structural restraints in the query, the user may still not retrieve the most valuable information.

As an example, consider the chemical equation below depicting a query that a user may present to a conventional database. The query uses a substructure. Substructures are fragments that define a core structural motif for which the user wishes

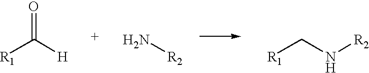

to search. In this example, the query specifies, an aldehyde fragment added to an amine fragment to yield a product. The chemical reaction is a reductive amination. As mentioned, with conventional databases, a query of this type would return every reaction in the database (sometimes several hundred or more) that conformed to the substructure fragments drawn. If a shorter list is desired, the user would have to submit a more constrained query in which the structures are more fully defined. Having used a more constrained structural query, the user is left with a more manageable list of reactions. However, this list describes only those particular literature reactions that have been loaded into the database. Thus, the user may be missing potentially valuable reaction data.

By utilizing a database of chemical reactions classified by type and of chemical reagents classified according to functional group and compatible synthetic methods, this invention can provide not only the aforementioned literature example reaction lists but also can generate examples based on literature precedent. This provides the user with variations (diversity) that perhaps were not considered, even if the user is an experienced chemist.

Further, because the database does not treat individual reaction transformations as static facts of reactivity, the general patterns of reactivity can be used to guide new products for synthesis. In addition to using precursors/building blocks as directly described in the literature, one may add additional building blocks from at least three separate sources. First, precursors/building blocks from the products of any other reaction in the database or other reactions added to the database. This creates a relational database based on actual known chemistries, as opposed to a static hierarchal database. Second, precursors/building blocks can be from files of molecules that can be important from any other sources (for example, all secondary amines from the Available Chemicals Directory). Third, precursors/building blocks can be drawn out by hand and imported into any reaction limited only by the imagination of the chemist working with the invention.

Note that the ability to capture diverse information in an integrated format with synthesis and enumeration capability allows for the generation of custom filters. The system can then evolve through enumeration to explore synthetically feasible molecules coupled to various customizable filters to rapidly identify desired functional molecules.

Chemical Diversity

Methods of the invention are embodied in software tools for generating diverse reaction sets, based on inputs from a user. As mentioned, the input may be a particular compound or class of compounds, or a particular reaction or class of reactions, for example.

The diverse reaction sets can provide a plurality of chemical compounds that are very likely to be synthetically accessible. This is because the invention uses databases in which complete synthetic pathways (sometimes referred to as reaction schemes) as represented in the literature, are broken into the individual reactions that comprise the larger pathway. These individual reactions are separately stored and intimately indexed in databases. This is unlike the situation with conventional chemical databases, where only complete syntheses, as reported in the literature, populate the databases. The present invention provides a more granular representation of chemical reactions. In this manner, the databases of this invention facilitate mixing and matching of individual chemical reaction steps to create new synthetic pathway. Thus, in some embodiments, the logic of the invention facilitates generation of novel synthesis schemes (and thus distinct molecule products) from literature precedents.

In some database designs of this invention, complete synthetic pathways (sometimes referred to as reaction schemes) as represented in the literature, are broken into the individual reactions that comprise the larger pathway. These individual reactions are separately stored or indexed in databases. This is unlike the situation with conventional chemical databases, where only complete syntheses, as reported in the literature, populate the databases. The present invention provides a more granular representation of chemical reactions. In this manner, the databases of this invention facilitate mixing and matching of individual chemical reaction steps to create new synthetic pathway. Thus, in some embodiments, the logic of the invention facilitates generation of novel synthesis schemes from the literature precedents.

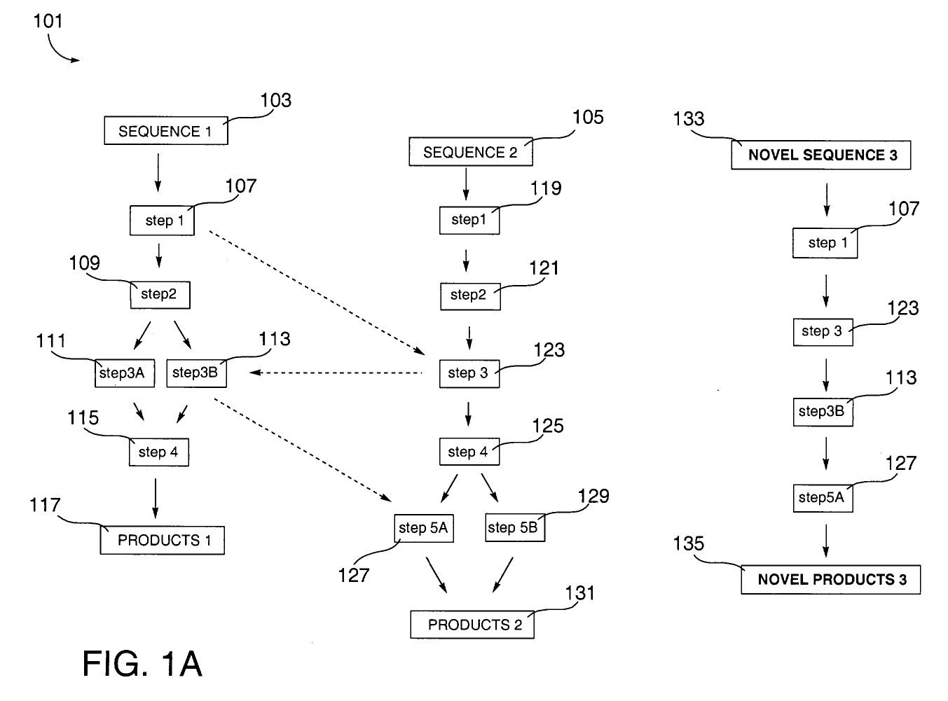

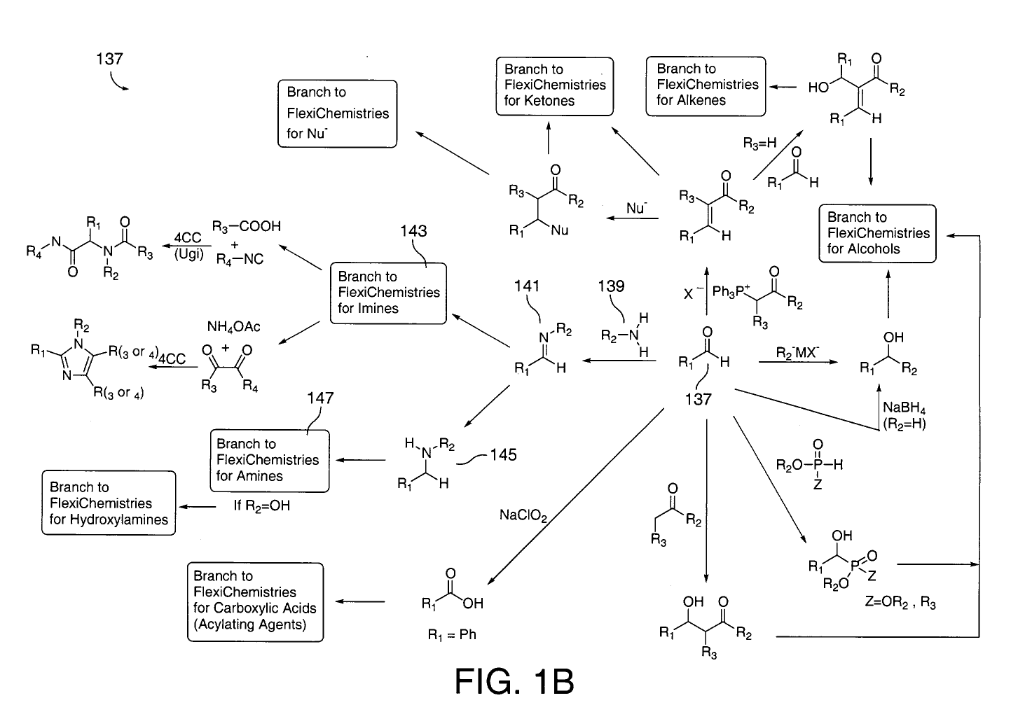

FIG. 1A is a block diagram 101 depicting how the logic of the invention may use literature precedent to generate a novel reaction sequence. Reaction sequences 103 and 105 are two examples of synthesis procedures taken from the literature and characterized in the database of the invention by the discrete reaction steps of which each consists. Each step is characterized by a unique set of conditions used to carry out that step. Sequence 103 consists of the individual steps 107-115 to give products 117. Likewise, sequence 105 consists of the individual steps 119-129 to give products 131.

Using conventional databases, given the appropriate query or queries, the user may be provided with individual steps (reactions) of reaction sequences 103 and 105. Although often times reaction sequences are not provided in discrete steps, but rather with a reactant, a product, and a conglomeration of text over an arrow describing two or more steps and associated process conditions. Since sequences in the databases of the invention are characterized by discrete steps and the steps are classified according to reaction type, the logic of the invention can use the steps to extrapolate from known sequences to generate novel sequences. As depicted by the dashed arrows, the logic of the invention can generate for example a new sequence 133, consisting of steps 107, 123, 113, and 127. This new sequence is generated using a “mix and match” algorithm, providing novel products 135. Many novel sequences can be generated from the many thousands of known chemical conversions in the literature. Also, a user can further massage and refine chemical information provided by the invention by application of filters, for example by specific process conditions, reliability ratings, pharmacokinetic parameters, and others.

The invention also finds particular use in parallel synthesis, in that it identifies a large number of divergent synthesis protocols for a particular reagent class query. FIG. 1B depicts a system of synthetic schemes 137. The logic employed by this invention provides various reaction schemes to users automatically. Thus, the user gains access to numerous reaction sequences for maximizing diversity. For example, a generic aldehyde 138 is input as a starting reaction class. The logic of the invention generates suitable synthetic pathways for reaction of 138 to make products. In one case, aldehyde 138 is reacted with amine 139 to give imine 141. This is but one reaction branch from aldehyde 138. As shown, however, multiple reactions may be generated from the starting aldehyde 138 to yield diverse products 149. Each of these products (149 and 141) is one reaction level removed from aldehyde 138. Some or all of these compounds can be further reacted to produce even more products. For example, imine product 141 is now used as a starting reagent for chemical reactions suitable to imines, 143. Further, imine 141 can be reduced to amine 145. Amine 145 represents a set of products two steps from aldehyde 138. Likewise, amine 145 is reacted further in chemical reactions suitable to amines, see 147. These linear and branched outward growth synthesis protocols create a large diverse pool of chemical compounds, all derived from aldehyde 138. Another level of diversity stems from the fact that aldehyde 138 represents a class of aldehydes; that is, each member of that class will produce a unique product for each reaction pathway to which it is exposed. Moreover, all products resulting from and reactants used with 138 also represent classes of compounds.

As depicted, many branch points are available from any single class of reactants. Not shown is yet another level of diversity created when novel and chemically diverse intermediates and products from the reactions depicted are themselves used as starting reactants in subsequent synthesis protocols with aldehyde 138 (and other intermediates along the pathways where suitable). For example amine 145, which was synthesized from 138, could be reacted with aldehyde 138. This provides a feedback diversity level.

Yet another level of diversity includes varying the reaction conditions where suitable in the above synthesis protocols. For example a particular reaction may provide a preponderance of a different product, depending on the time allowed and temperature applied. Thus characterizing reactions in discrete steps as described above allows the logic of the invention to maximize diversity by mixing and matching reactants, intermediates, and products with synthetic steps and reaction conditions. Importantly, reliability ratings (assigned to for example reaction conditions, reaction types, starting materials, reactants, and reagents) give the methods of the invention added value, in that the user has data concerning the likelihood that a given sequence will work.

Process Flow

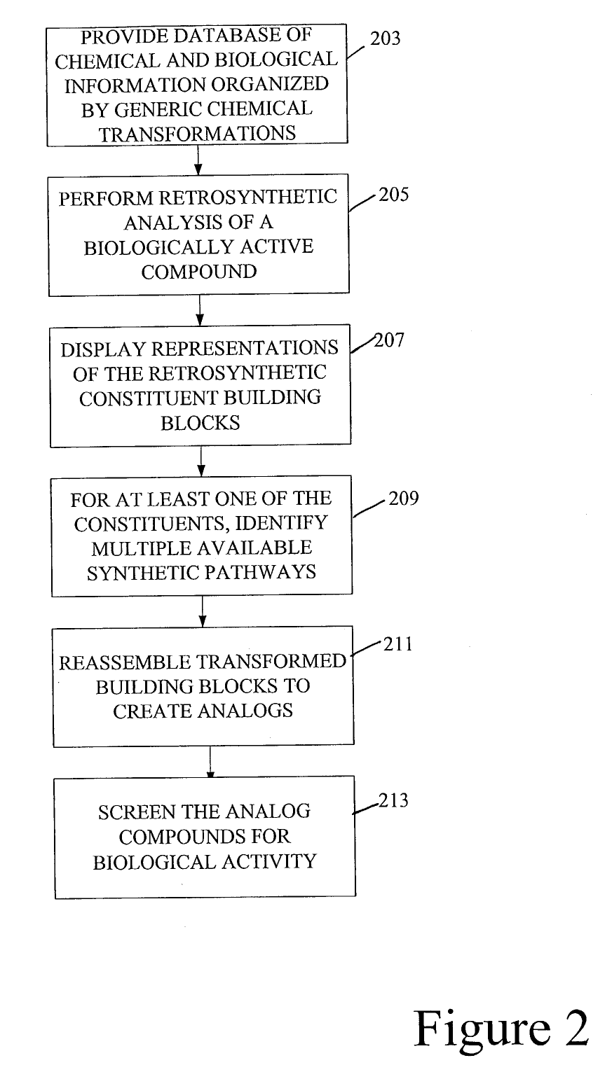

FIG. 2 depicts a computational method and associated data arrangement for identifying a relationship between biological activity and one or more chemical features by using a database of this invention. As depicted in FIG. 2, one example of a process flow of this invention begins at a process block 203 with provision of a database containing chemical and biological information organized by generic chemical transformations. As indicated above, chemical transformations may involve chemical reactions and/or chemical protocols. Thus, chemical transformations include reactants, products, and sometimes intermediates. In their generic format, at least some compounds of the transformations are represented with generic Markush R groups. The database in question provides information keyed to specific chemical compounds—as well as generic chemical transformations. Note that within the database many of the specific chemical compounds may be associated with one or more of the chemical transformations. Further, the database associates at least some of the specific chemical compounds with biological information. Such information may take the form of an activity value representing interaction with one or more biological molecules. Still further, the database may associate chemical compounds with particular chemotypes or substructures contained therein.

Next at 205, the computation process performs a retrosynthetic analysis of a particular biologically active compound identified by the user. This analysis may be performed entirely by computation or together with a user's input. In some cases, the biologically active compound is identified in the database by one or more reported synthetic pathways. The computational method can identify the reactants used in these pathways as part of the retrosynthetic analysis. The reactants are treated as components or constituents of the starting compound and they are available for further flexible analysis and assembly into analogs of the starting compound. Other mechanisms for identifying constituent compounds from the starting compound will be known to those of skill in the art. Some such mechanisms involve computationally parsing a structural representation of the compound, such as a mark up language representation.

At 207, each of the various constituent compounds identified by retrosynthetic analysis is optionally displayed for the user. The display may present the compounds in fully elaborated format or in Markush format. Typically, the system displays the compounds via a user interface. Next, at a computational process operation 209, the control logic identifies multiple available synthetic pathways for at least one of the constituents identified by the retrosynthetic analysis. This operation may be performed with the aid of user input to focus on certain synthetic pathways or automatic selection in computational apparatus. Automatic selection may be based on criteria such as ease of reaction, available reagents or reaction conditions, stability of products, etc. The above discussion presents examples of how a single constituent or class of constituents can undergo multiple reactions.

At block 211, the computational system chemically links some or all of the various transformed building blocks (generated at 209) to create molecules comparable in size and/or overall layout to the original biologically active compound. The chemical linkages should be chosen to present certain features of the original molecule. For example, they may be provided so as to present certain moieties, or more generically functional types, at orientations comparable those found in the original compound. As a specific example, the linkage may be conducted in a manner that presents a hydrophobic region at a first location, a hydrogen bond donor at a second location separated from the first location by a certain number of angstroms, and a nitrogen-containing aromatic group at a third location separated by the first and second locations by specified angular ranges and distances. Alternatively, the transformed building blocks can be linked arbitrarily and then filtered by a pharmacophore filter, for example, to remove those products that are sufficiently dissimilar to the original compound.

Finally at 213, computational screens are used to identify products generated at 211 that likely result in a biological activity of interest. Various algorithms with various thresholds may be employed to screen the computationally derived products of interest. For example, the computational system may predict binding values (e.g., Ki) for targets of interest. These values may be predicted by a pharmacophore matching calculations, for example. In another example, binding values are predicted based upon calculations with a docking algorithm. Still another example involves using ADME screens.

Frequently the biological activity values related to an interaction with at least one biological molecule such as a receptor or enzyme. More specifically, the biological activity value may represent binding with a target, a class of targets, a binding site on a target, or binding sites of a class of targets. Examples of such values include IC50, Ki, Km, and mean days of survival.

A final operation of interest in the exemplary process flow involves selecting one or more compounds for further investigation. These compounds are selected based upon the relationship identified or derived at block 213. They may be screened in vitro, and, if appropriate, investigated further as pharmaceutical candidates. In a related approach, the selected compounds comprise at least part of a primary or secondary library of chemical compounds.

### EXAMPLES

### Example 1

The following example illustrates how one can employ a database and method of this invention. The goal of this example is to identify new therapeutic equivalents to the leukemia drug, Gleevec™ (imatinib mesylate) marketed by Novartis Pharmaceuticals Corporation of East Hanover, N.J.

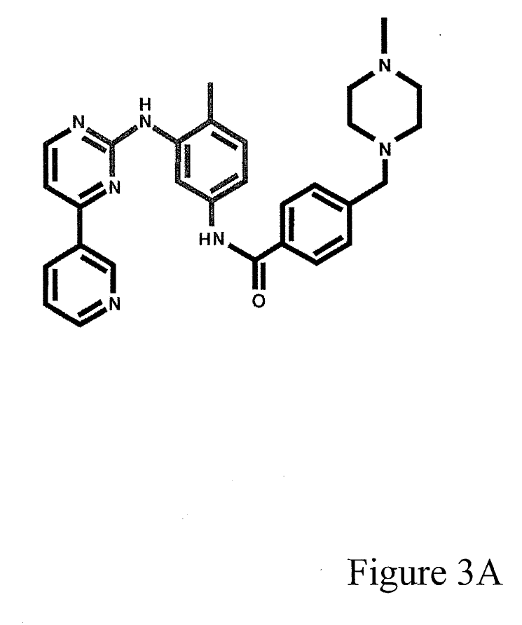

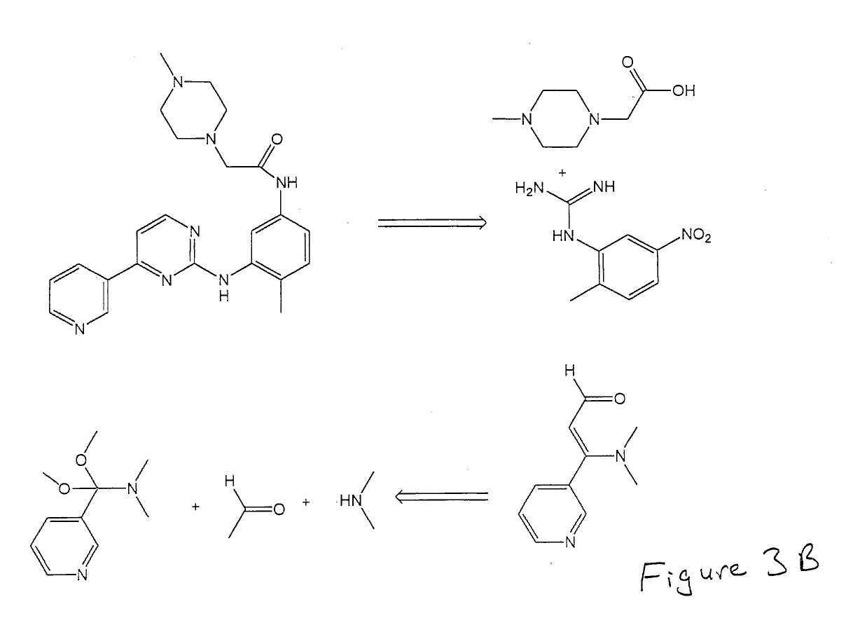

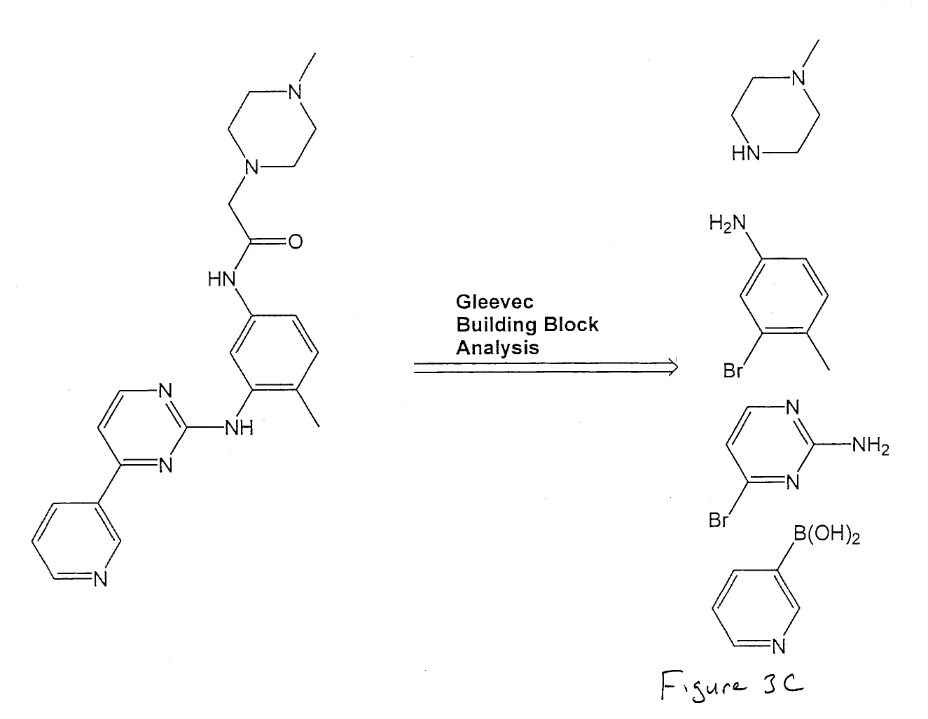

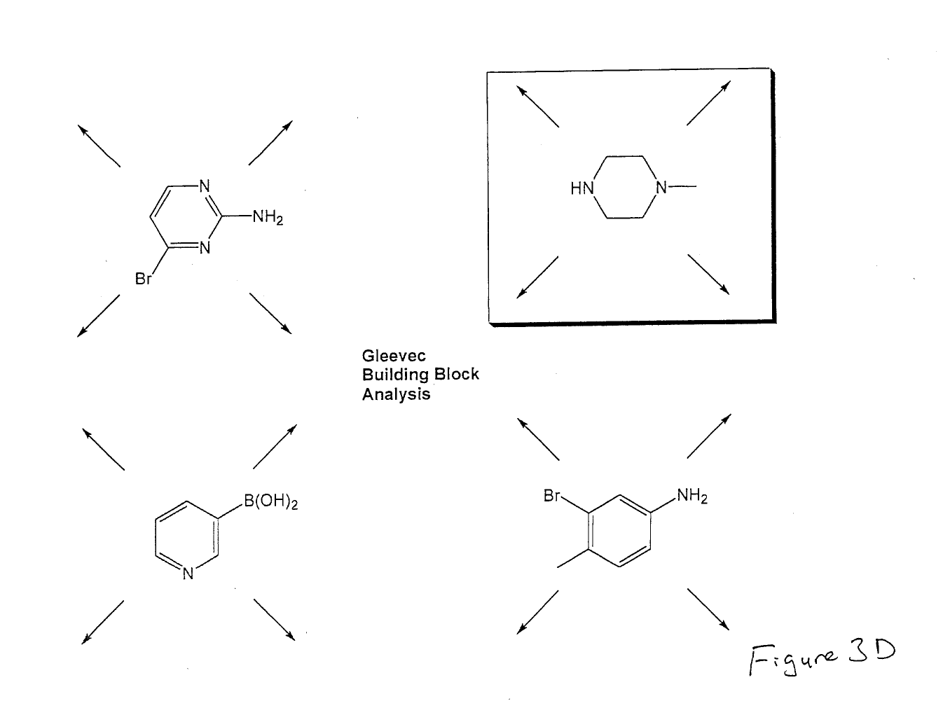

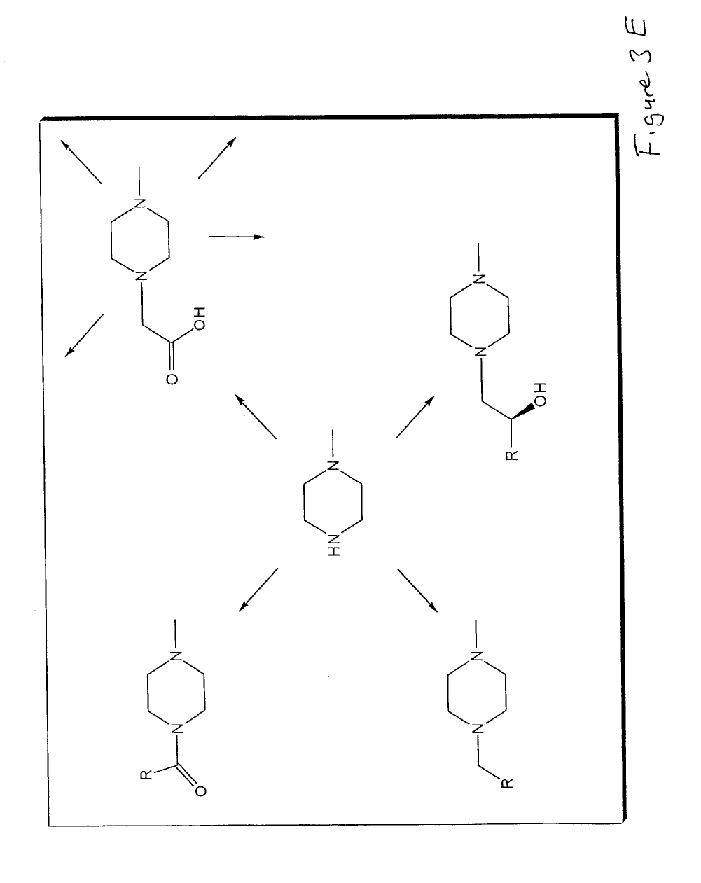

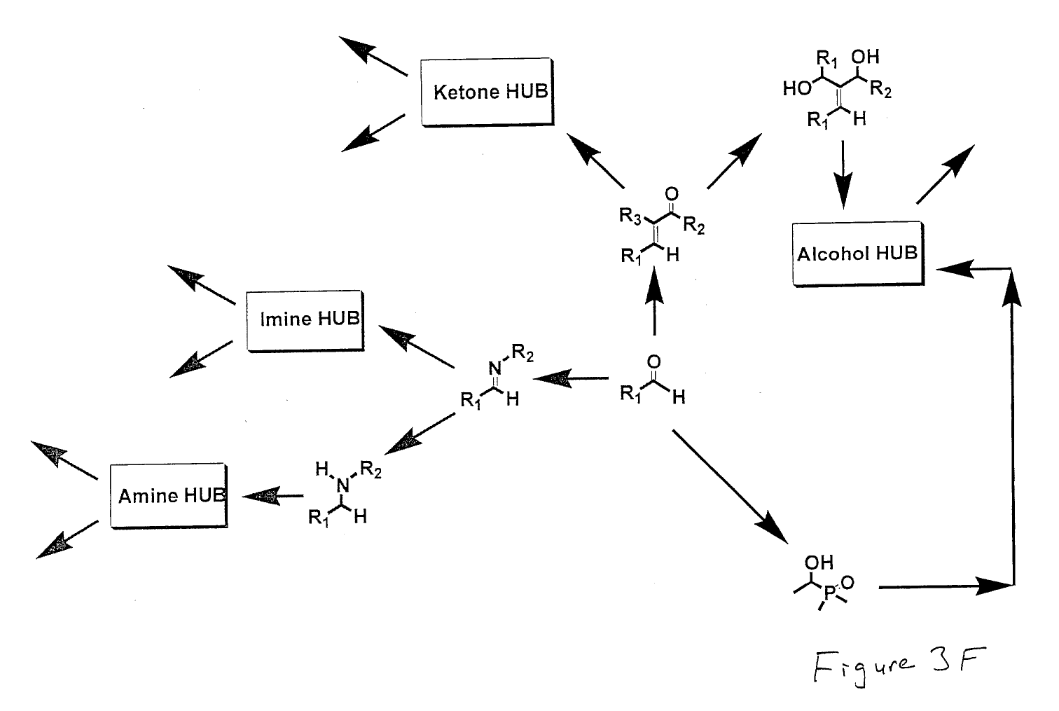

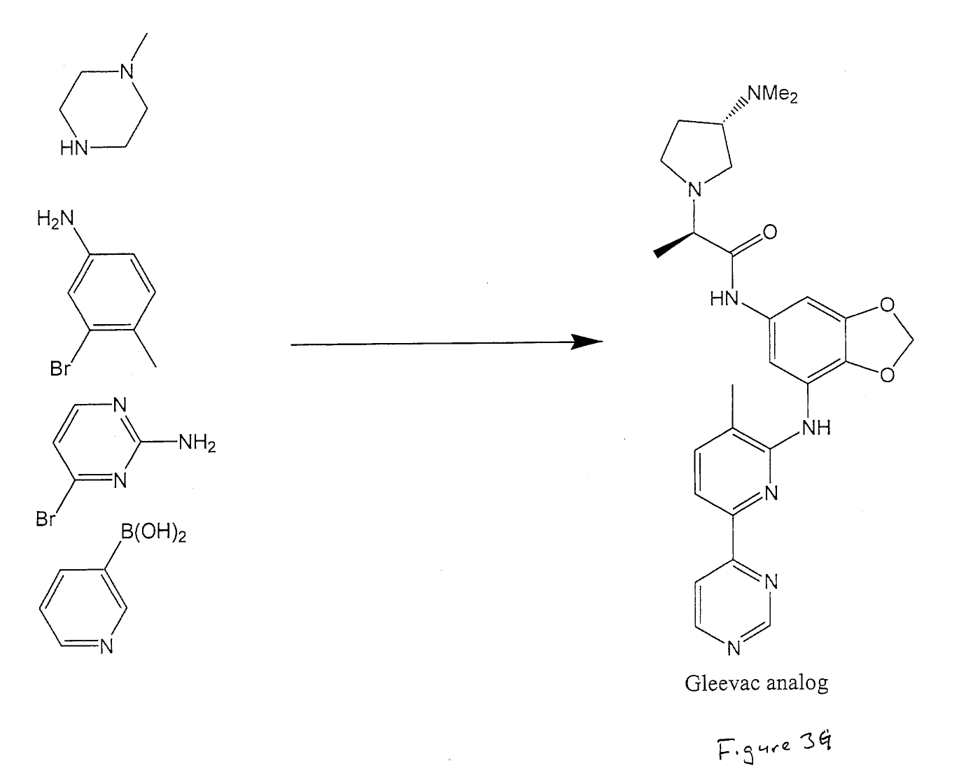

The chemical structure of Gleevec™ is used as a starting point. This structure is shown in FIG. 3A. The initial goal is to perform a retrosynthetic analysis that identifies molecular components that might serve as starting points to develop analogs that could have activity similar to Gleevec™. Chemical space is explored by proposing various acceptable reactions of the Gleevec™ components. The resulting reaction products are enumerated and screened computationally. But initially, there must be some mechanism for identifying constituent parts of Gleevec™.

To this end, one may use a markup language such as SMILES (Daylight Chemical Information Systems, Inc.) to parse molecule into its component parts. The use of such markup languages is widely known to those of skill in the art and is described in U.S. provisional patent application No. 60/359,643, filed Feb. 22, 2002 by Schurer et al., and incorporated herein by reference for all purposes. Preferably, the parsing follows a synthesis route or multiple synthesis routes reported in the literature or otherwise known and appearing in a database of this invention. Because the database stores information in the form of synthetic pathways, the reactants employed to synthesize Gleevec™ are also stored in the database. These are identified by appropriate database queries and serve as the results of a retrosynthetic analysis. FIGS. 3B-3C depict, structurally, a suitable retrosynthetic analysis performed on the Gleevec™ molecule.

With the constituents chosen, one can use a pathway recombination analysis method to build up larger molecular structures that potentially exhibit properties similar to (or improved over) those of the original compound (Gleevec™ in this example). Multiple available synthetic pathways are selected by the software in the manner depicted in FIGS. 3D and 3E. FIG. 3D shows each of four Gleevec™ building blocks that can be elaborated using various synthetic pathways. FIG. 3E shows specific elaboration of one of these building blocks (a piperazine) to produce a number of transformed building blocks. The software may select all or a subset of the synthetic pathways available to a starting component depending upon whether chemical or biological filters are set. FIG. 3F depicts, for the sake of illustration, the various synthetic pathways that are available to a generic aldehyde constituent.

Generally, to select and analyze various synthetic reaction pathways, the software (with or without aid from a user) must start with either a starting material building block or an intermediate that could be used as a precursor in a reaction. The software can then consider various specific reactions to specific products. Every instance of that occurrence in the knowledgebase/database or an instance added by an end user can be used as a potential reaction transformation. Another approach involves use of a Markush representation of the building block/precursors. In this case, confidence in the likelihood of success in the reaction can be increased by using the database to look for the patterns of the building blocks that do (and those that do not) successfully react. Finally, if one does not know a priori what the optimal reaction or product would be for a particular problem, then the software (with or without aid from a user) can evaluate a number of different, complementary reactions that give rise to diverse products all originating from the same starting material or intermediate. Of course, these are products found in the knowledgebase/database with their associate synthetic reactions.

As part of the flexible transformation process, the software optionally searches the chemical database for molecular analogs matching the transformed building blocks identified by computationally applying various synthetic routes as previously discussed. This operation may be implemented as an enumeration process as described above. That is, the generic moieties “R” are converted to specific moieties (e.g., —NH(Et)) to provide a list of specific molecules.

The transformed building blocks are then linked to one another at positions selected to produce analogs of the original Gleevec™ molecule. See FIG. 3G for an example. Because the software contains a structural representation of the original Gleevec™ molecule, it can favor or require assembly of the enumerated components designed to preserve, to some degree, the three dimensional arrangement of moieties in the original molecule.

In cases where a specific molecule like Gleevec™ is desired, the software can identify both “exact hits” for synthesis and “related hits” that would have a reasonable probability of working. In one example, the software does a tanimoto similarity search of enumerated products (based on pharmacophore matching) and prioritizes/organizes them according to similar 3-D structures. See PCT application WO 00/25106, published Apr. 4, 2000, which is incorporated herein by reference for all purposes. In cases where the specific molecule is not required, but rather where the general type of structure or structural class is deemed to be desirable then the technology can use a variety of general reactions to build similar novel structures without regard to structural matching.

More generally, the software uses algorithms to prioritize pathways for analog construction. Examples of other such algorithms include algorithms based on reported reaction yield, reaction condition constraints (time, temperature, etc.), stereochemical constraints, and the like. Finally, the software may apply relevant biological filters to screen out therapeutically compromised molecules for later synthesis.

### Example 2

The following is a description of a working example employing a database and method of this invention. The goal of this example was to design therapeutic alternatives to the leukemia drug, Gleevec™ (imatinib mesylate). The work used information about the protein target, Abl kinase. As indicated, the chemical structure of Gleevec™ is shown in FIG. 3A.

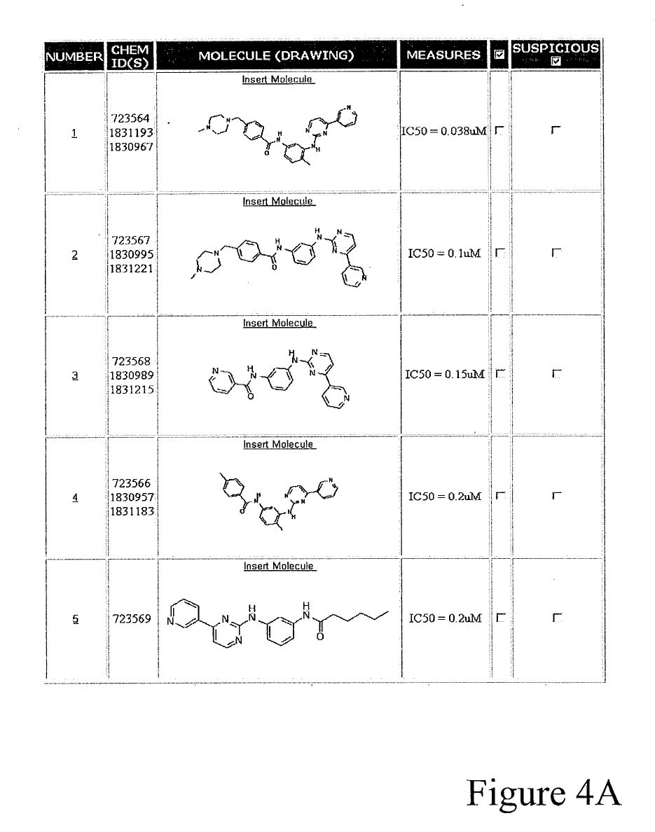

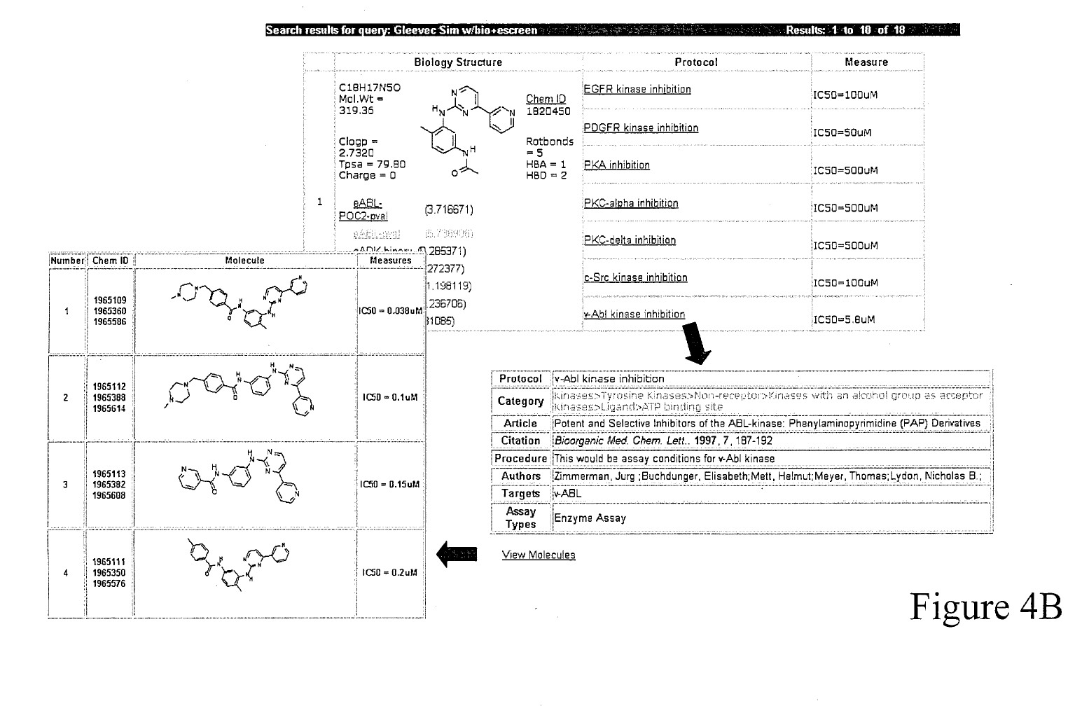

The chemical database employed in this example included data from numerous literature sources. The data was captured and abstracted into the database. Specifically, the data included reaction pathways, structures, and biological activities. In many cases, the data associated with a particular organic compound included synthesis information and/or biological information (e.g. IC50 values). FIG. 4A shows a sample screen shot from a database application used with this example.

Next, molecules with IC50's were used to develop a specific electronic screen for Abl Kinase. This screen was pharmacophore based predictive model that predicted interaction with Abl Kinase based upon chemical structure. The starting point for generating this model was training sets developed using SARs (Structure-Activity Relationships) from both public domain articles and patents for small molecule Abl Kinase inhibitors. Pharmacophore fingerprints (see published PCT application WO 00/25106) were determined for each molecule. Here, molecular conformations were explored through the rotation of all flexible dihedrals (flexible ring systems are considered as well). For each conformation, the presence/absence of any of ten thousand different 3-point pharmacophores was identified and placed into a fingerprint (based on seven pharmacophore types including +/− charge, H-bond donors/acceptors, hydrophobic/aromatics, or any other and six distance ranges from 2-4.5, 4.5-7, 7-10, 10-14, 14-19, and 19-24 angstroms between the various pharmacophores in the molecules), for example. Note that the methods could be used with any of a number of prioritization filters (e.g. similarity, QSAR models, docking models, MW rankings, etc.). The model generation process is depicted graphically in FIG. 4B.

The data used in this example contained information on approximately 2,060,000 compounds, about 1,000,000 of where provided in a database of this invention. Three separate scaffolds were chosen based on analysis of the Gleevec™ molecule. With the aid of the database, researchers identified various reaction chemistries available to those scaffolds. The database was then used to enumerate 60,000 novel analogs as described above. Each of these compounds was screened using the electronic pharmacophore screen described above. The top 120 compounds identified from this screening were then selected for actual synthesis and wetlab bioassay. From these compounds, researchers identified a novel lead series with potency approaching Gleevec™. Specifically, ten of the compounds showed ≦10 μM activity, and six had single-digit μM activity. Compounds discovered by this method are the subject of a pending provisional patent application: U.S. provisional patent application No. 60/400,828, filed Aug. 2, 2002 by Powers et al., and incorporated herein by reference.

### Example 3

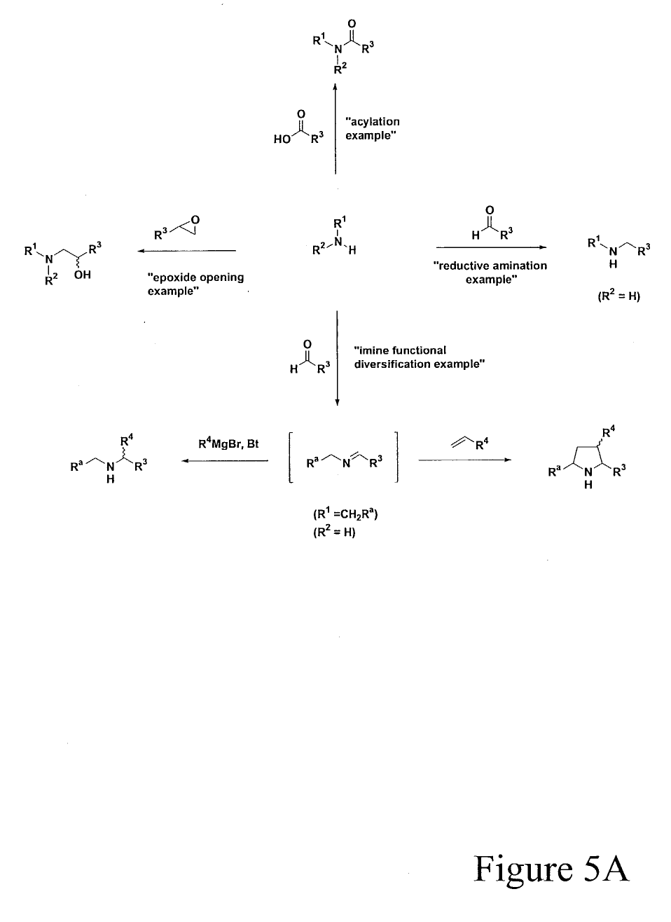

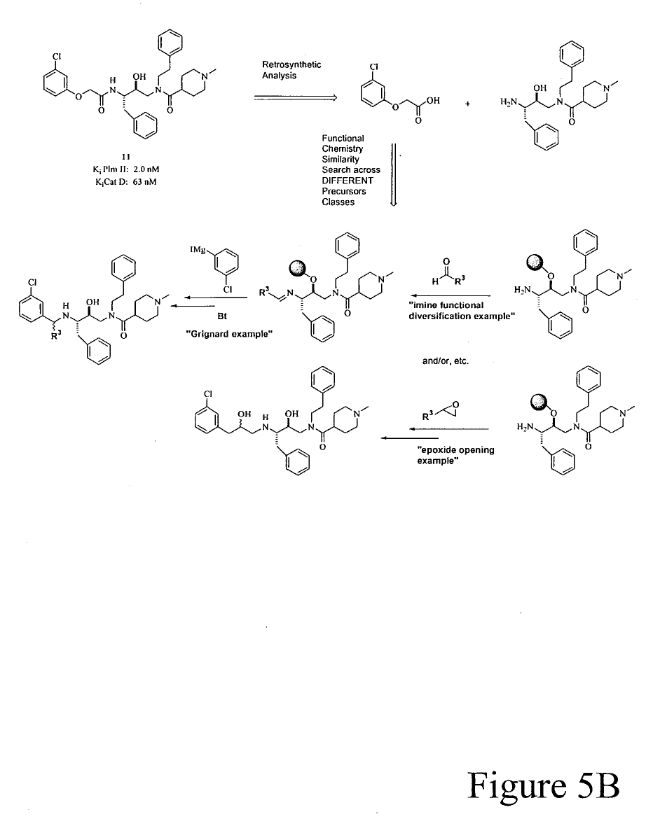

Discovering replacements for the amide bond in the statine-based inhibitors is a problem that demonstrates the methods of the invention implemented in software form. First, consider FIG. 5A, which illustrates how the invention uses databases (organized as described above) for example to elaborate a generic group of building blocks around the common amine functional group. After exploring the chemical space accessible from the amine (which happens to be a disconnect in the retrosynthetic analysis of the Plasmepsin II inhibitors), one can see how the technology would be applied to the rapid identification of novel pharmacophores and chemotypes.

Many synthetic routes can rapidly and cost-effectively be generated in the computer from known, experimentally validated methods. After generating possible chemical pathways computationally, one can then prioritize the products based on various appropriate filters such as structural similarity, cost of synthesis/precursors, SAR patterns, and Lipinski-type filters to predict PK/ADME properties, etc. A major differentiation from conventional rational design efforts is that the invention starts with a known set of compounds and synthetic routes defined by actual experimental results published by the chemistry community throughout the world (and later the broader synthetic community).

The invention makes use of a vast array of chemistry that can be done with a particular functional group. After examining the synthetically accessible routes in the computer, one can do a similarity search to known inhibitors but with a library derived from multiple different chemistries. This then allows one to explore all possibilities with maximal efficiency and creativity to discover fundamentally new chemotypes. In one example, the goal is simply to replace the amide bond with a functional group that is different. The peptidomimetic structures with amide bonds could potentially suffer from the traditional liability of peptides as therapeutic agents (low half-life in the bloodstream, hydrolysis by proteases, and rapid excretion). A primary goal of peptidomimetic design has been to replace the amide with another functional group while retaining activity. The utility of the invention within the context of replacing the amide bond of the statine-based inhibitor is shown below in FIG. 5B.

In one case the amide has been replaced by an amino-alcohol by opening up a judiciously selected epoxide (only the nearest match shown). In the second case, two reactions are used in conjunction: imine formation with an aldehyde followed by the addition of a Grignard reagent in the presence of benzotriazole. These examples elude to the scope and limitations of the methodology. For example, the Griggs 2+3 cyclization would not work in this context because of the absence of an acidic proton alpha to the amine. Initially the invention will provide options requiring human intelligence to filter as appropriate. Over time, as the rules that govern synthetic reactivity are better understood and captured in the database format the intelligence and predictability of the system will increase.

To be a true expert system for synthetic chemistry, the database must avoid doing “unrealistic” chemistry that will not work in the laboratory due to functional group incompatibility or steric and electronic factors. Because the system is constantly enriched with experimental data based on chemistries reported to work with particular sets of precursors throughout the literature, the information to avoid “unrealistic” chemistry is embedded in the dataset. With enough data one can develop a predictability rating for certain chemistries in novel contexts based on a careful statistical analysis and grouping the reagents and transformation into similar sets.

Software/Hardware

Generally, embodiments of the present invention employ various processes or methods involving data stored in or transferred through one or more computing devices. Embodiments of the present invention also relate to an apparatus for performing these operations. This apparatus may be specially constructed for the required purposes, or it may be a general-purpose device (e.g., a computer) selectively activated or reconfigured by a set of instructions (e.g., a computer program) and/or data structure provided to the apparatus. The processes presented herein are not inherently related to any particular computer or other apparatus. In particular, various general-purpose machines may be used with programs written in accordance with the teachings herein, or it may be more convenient to construct a more specialized apparatus to perform the required method steps. A particular structure generally representing a variety of these machines will be described below.

In addition, embodiments of the present invention relate to computer readable media or computer program products that include program instructions and/or data (including data structures) for performing various computer-implemented operations. Examples of computer-readable media include, but are not limited to, magnetic media such as hard disks, floppy disks, and magnetic tape; optical media such as CD-ROM disks; magneto-optical media; semiconductor memory devices, and hardware devices that are specially configured to store and perform program instructions, such as read-only memory devices (ROM) and random access memory (RAM). The data and program instructions of this invention may also be embodied on a carrier wave or other transport medium (including electronic or optically conductive pathways).

Examples of program instructions include both machine code, such as produced by a compiler, and files containing higher level code that may be executed by the computer using an interpreter. Further, the program instructions include machine code, source code and any other code that directly or indirectly controls operation of a computing machine in accordance with this invention. The code may specify input, output, calculations, conditionals, branches, iterative loops, etc.

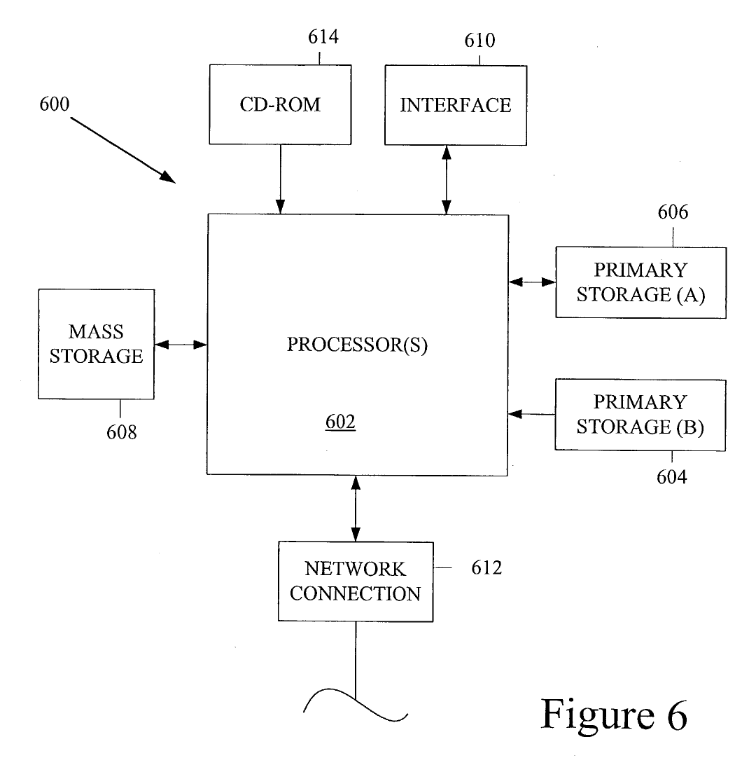

FIG. 6 illustrates, in simple block format, a typical computer system that, when appropriately configured or designed, can serve as a computational apparatus of this invention. The computer system 600 includes any number of processors 602 (also referred to as central processing units, or CPUs) that are coupled to storage devices including primary storage 606 (typically a random access memory, or RAM), primary storage 604 (typically a read only memory, or ROM). CPU 602 may be of various types including microcontrollers and microprocessors such as programmable devices (e.g., CPLDs and FPGAs) and unprogrammable devices such as gate array ASICs or general purpose microprocessors. As is well known in the art, primary storage 604 acts to transfer data and instructions uni-directionally to the CPU and primary storage 606 is used typically to transfer data and instructions in a bi-directional manner. Both of these primary storage devices may include any suitable computer-readable media such as those described above. A mass storage device 608 is also coupled bi-directionally to CPU 602 and provides additional data storage capacity and may include any of the computer-readable media described above. Mass storage device 608 may be used to store programs, data and the like and is typically a secondary storage medium such as a hard disk. It will be appreciated that the information retained within the mass storage device 608, may, in appropriate cases, be incorporated in standard fashion as part of primary storage 606 as virtual memory. A specific mass storage device such as a CD-ROM 614 may also pass data uni-directionally to the CPU.

CPU 602 is also coupled to an interface 610 that connects to one or more input/output devices such as such as video monitors, track balls, mice, keyboards, microphones, touch-sensitive displays, transducer card readers, magnetic or paper tape readers, tablets, styluses, voice or handwriting recognizers, or other well-known input devices such as, of course, other computers. Finally, CPU 602 optionally may be coupled to an external device such as a database or a computer or telecommunications network using an external connection as shown generally at 612. With such a connection, it is contemplated that the CPU might receive information from the network, or might output information to the network in the course of performing the method steps described herein.

In one embodiment, the computer system 600 is configured as a database and database management system for chemical information organized as described herein. The chemical information may derive from various sources. Remote sources of chemical information may provide the information to system 600 via interface 612.

Once in the apparatus 600, a memory device such as primary storage 606 or mass storage 608 stores the chemical information. The memory may also store various routines and/or programs for analyzing and presenting the data. Such programs/routines may include database management systems, search engines, filtering programs (including QSAR programs, docking programs, ADME property prediction programs, etc.) programs for populating databases with new chemical information, tools for improving the performance of databases, etc.

Other Embodiments

While this invention has been described in terms of a few preferred embodiments, it should not be limited to the specifics presented above. Many variations on the above-described preferred embodiments may be employed. Therefore, the invention should be broadly interpreted with reference to the following claims.

What is claimed is:

1. On a computing device, a method of identifying, from a database, a collection of chemical compounds that can be synthesized from a common reactant or class of reactants, the method comprising:
(a) automatically generating a list of reactions that the class to which the reactant or reactants belong undergoes; and
(b) for each reaction in the list, identifying a product or class of products, which products comprise the collection of chemical compounds, wherein at least some of the products are identified in the database, which contains reactions reported in references, and at least some of the products are identified without reliance on reactions reported in references.

2. The method of claim 1, further comprising:
(c) treating at least one of the products as a new reactant;
(d) automatically generating a new list of reactions that the class to which the new reactant belongs; and
(e) for each reaction in the new list, identifying a new product or class of products produced by the reaction, wherein the new products are added to the collection of compounds.

3. The method of claim 1, further comprising:
(c) filtering the products identified at (b) based on one or more of the following criteria: predicted activity and specified reaction conditions.

4. The method of claim 1, wherein generating the list of reactions is accomplished, at least in part, without reliance on reactions reported in references.

5. On a computing device, a method of identifying a collection of chemical compounds that can be synthesized from a common reactant or class of reactants, the method comprising:
(a) automatically generating a list of reactions that the class to which the reactant or reactants belong undergoes;
(b) for each reaction in the list, identifying a product or class of products; and
(c) filtering the products identified in (b) to yield a subset of the compounds identified (b), which subset comprises the collection of chemical compounds, wherein the filtering is based on one or more of the following criteria: predicted activity, reaction reliability rating, reactant reactivity reliability rating, reactant reactivity profile, number of reaction steps required to produce the product, and specified reaction conditions.

6. The method of claim 5, wherein at least some of the products are identified in a database containing reactions reported in references and at least some of the products are identified without reliance on reactions reported in references.

7. The method of claim 5, wherein generating the list of reactions is accomplished, at least in part, without reliance on reactions reported in references.

8. The method of claim 5, further comprising:
(d) treating at least one of the compounds in the subset as a new reactant;
(e) automatically generating a new list of reactions that the class to which the new reactant belongs; and
(f) for each reaction in the new list, identifying a new product or class of products produced by the reaction, wherein the new products are added to the collection of compounds.

9. A computer program product comprising a machine readable medium on which is provided program instructions for identifying, from a database, a collection of chemical compounds that can be synthesized from a common reactant or class of reactants, the program instructions comprising:
(a) code for automatically generating a list of reactions that the class to which the reactant or reactants belong undergoes; and
(b) code for identifying a product or class of products, for each reaction in the list, which products comprise the collection of chemical compounds, wherein at least some of the products are identified in the database, which contains reactions reported in references, and at least some of the products are identified without reliance on reactions reported in references.

10. The computer program product of claim 9, wherein the program instructions further comprise:
(c) code for treating at least one of the products as a new reactant;
(d) code for automatically generating a new list of reactions that the class to which the new reactant belongs; and
(e) code for identifying a new product or class of products produced by the reaction, for each reaction in the new list, wherein the new products are added to the collection of compounds.

11. The computer program product of claim 9, wherein the program instructions further comprise:
(c) code for filtering the products identified at (b) based on one or more of the following criteria: predicted activity and specified reaction conditions.

12. The computer program product of claim 9, wherein the code for generating the list of reactions can generate the list, at least in part, without reliance on reactions reported in references.

13. A computer program product comprising a machine readable medium on which is provided program instructions for identifying a collection of chemical compounds that can be synthesized from a common reactant or class of reactants, the program instructions comprising:
(a) code for automatically generating a list of reactions that the class to which the reactant or reactants belong undergoes;
(b) code for identifying a product or class of products for each reaction in the list; and
(c) code for filtering the products identified in (b) to yield a subset of the compounds identified (b), which subset comprises the collection of chemical compounds,
wherein the filtering is based on one or more of the following criteria: predicted activity, reaction reliability rating, reactant reactivity reliability rating, reactant reactivity profile, number of reaction steps required to produce the product, and specified reaction conditions.

14. The computer program product of claim 13, wherein the code for identifying products is written to identify at least some of the products in a database containing reactions reported in references and to identify at least some of the products without reliance on reactions reported in references.

15. The computer program product of claim 13, wherein the code for generating the list of reactions is written to generate the list, at least in part, without reliance on reactions reported in references.

16. The computer program product of claim 13, further comprising:
(d) code for treating at least one of the compounds in the subset as a new reactant;
(e) code for automatically generating a new list of reactions that the class to which the new reactant belongs; and
(f) code for identifying, for each reaction in the new list, a new product or class of products produced by the reaction, wherein the new products are added to the collection of compounds.

17. A computer implemented method of identifying biologically active compounds based on the chemical structure of a known biologically active compound, the method comprising:
(a) retrosynthetically decomposing the known biologically active compound into two or more building blocks;
(b) identifying multiple chemical reactions that a first one of the building blocks can undergo;
(c) identifying, with the aid of a chemical database, products of said multiple chemical reactions;
(d) identifying a potentially biologically active compound by linking at least one of said products with one or more of the other building blocks, whether transformed or not; and
(e) conducting a computational screen to predict whether the potentially biologically active compound is likely to be biologically active.

18. The method of claim 17, further comprising:
identifying a second set of chemical reaction that a second one of the building blocks can undergo; and
identifying, with the aid of the chemical database, products of the second building block when subjected to the second set of chemical reactions, wherein the potentially biologically active compound is created by linking a product of the first building block with a product from the second building block.

19. The method of claim 17, wherein the screen is a pharmacophore screen or a docking algorithm.

20. A computer program product comprising a machine readable medium on which is provided program instructions for identifying biologically active compounds based on the chemical structure of a known biologically active compound, the program instructions comprising:
(a) code for retrosynthetically decomposing the known biologically active compound into two or more building blocks;
(b) code for identifying multiple chemical reactions that a first one of the building blocks can undergo;
(c) code for identifying, with the aid of a chemical database, products of said multiple chemical reactions;
(d) code for identifying a potentially biologically active compound by linking at least one of said products with one or more of the other building blocks, whether transformed or not; and
(e) code for conducting a computational screen to predict whether the potentially biologically active compound is likely to be biologically active.

21. The computer program product of claim 20, further comprising:
code for identifying a second set of chemical reaction that a second one of the building blocks can undergo; and
code for identifying, with the aid of the chemical database, products of the second building block when subjected to the second set of chemical reactions,
wherein the code for identifying a potentially biologically active compound is written to link a product of the first building block with a product from the second building block.

22. The computer program product of claim 20, wherein the screen is a pharmacophore screen or a docking algorithm.
